(19) United States
(12) Patent Application Publication
(10) Pub. No.: US 2024/0065989 A1
(43) Pub. Date: Feb. 29, 2024

(54) METHOD AND COMPOSITION FOR TREATING TREATMENT RESISTANT DEPRESSION

(71) Applicants: Taghogho Agarin, San Jose, CA (US); Michael Callis, Santa Maria, CA (US); Robert McLay, Chula Vista, CA (US)

(72) Inventors: Taghogho Agarin, San Jose, CA (US); Michael Callis, Santa Maria, CA (US); Robert McLay, Chula Vista, CA (US)

(73) Assignee: Zyvran Pharmaceuticals, San Jose, CA (US)

(21) Appl. No.: 18/220,569

(22) Filed: Jul. 11, 2023

Related U.S. Application Data
(60) Provisional application No. 63/402,868, filed on Aug. 31, 2022.

(51) Int. Cl. A61K 31/137; A61K 31/55; A61K 33/00

(52) U.S. Cl. CPC A61K 31/137; A61K 31/55; A61K 33/00

(57) ABSTRACT

A composition for the treatment of treatment resistant depression may include a serotonin and norepinephrine reuptake inhibitor (SNRI), such as venlafaxine; a tetracyclic antidepressant (TeCA), such as mirtazapine; and a mood stabilizer, such as lithium. A method of treating treatment resistant depression may include administering, to a patient in need, a therapeutically effective dosage of the composition.

### RELATED APPLICATION

This application claims priority to provisional patent application U.S. Ser. No. 63/402,868 filed on Aug. 31, 2022, the entire contents of which is herein incorporated by reference.

### BACKGROUND

The embodiments described herein relate generally to therapies and treatments for depression and, more particularly, to a treatment composition for treating treatment resistant depression.

As of 2015, 322 million people suffer from Major Depressive Disorder (MDD). Using objective scales, in research circles a treatment is considered nonresponsive if it achieves less than 22 total score on the Montgomery and Asberg Depression Rating Scale (MADRS) and less than 50% reduction in total MADRS score. If an individual has experienced two different non-responses, such is classified as treatment resistant depression (TRD).

Unfortunately, first line treatment is usually only effective in about a third of cases. In fact, one study of 1410 patients in a European naturalistic study showed adequate treatment response in only about 25% of cases. More specifically, studies have shown that only about 25% of patients with MDD have an adequate response to first-line antidepressant treatments, meaning that they have refractory or treatment-resistant depression. These patients tend to have a more severe course of illness and are at an increased risk of suicide.

Treatment options for patients with TRD include switching to a different antidepressant, adding another antidepressant to the regimen, or adding another non-antidepressant medication. Such a combination of a second-generation antipsychotic, such as olanzapine, and the selective serotonin reuptake inhibitor (SSRI) fluoxetine was one of the first pharmacotherapies approved for TRD. However, its use is limited due to significant metabolic side effects.

Other strategies include adding a mood stabilizer to a fire-line medication (e.g., an SSRI) with a lithium or with an anticonvulsant, such as lamotrigine. Other approaches include combining an SSRI with an atypical antidepressant, such as bupropion, or a serotonin and norepinephrine reuptake inhibitor (SNRI), such as Effexor, or an SSRI with tricyclics, such as imipramine, or with a tetracyclic antidepressant (TeCA), such as mirtazapine. Recently, drugs that are glutamate antagonists, such as esetamine or ketamine or the class of drugs, like adamantanes (e.g., amantadine) have been used for TRD and studies have suggested some success. They are N-methyl-D-aspartate (NMDA) receptor antagonists that raise dopamine levels. These classes of drugs may show some utility when a rapid onset of action against suicidal ideation is required.

However, comparting antidepressants and augmentation strategies on the basis of symptom relief, tolerability, and efficacy is not well established.

Therefore, what is needed is a method and composition for treating treatment resistant depression.

### SUMMARY

Some embodiments of the present disclosure include a composition for the treatment of treatment resistant depression. The composition may include a serotonin and norepinephrine reuptake inhibitor (SNRI), such as venlafaxine; a tetracyclic antidepressant (TeCA), such as mirtazapine; and a mood stabilizer, such as lithium. A method of treating treatment resistant depression may include administering, to a patient in need, a therapeutically effective dosage of the composition.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The composition of the present disclosure may be used as a single administration for treating treatment resistant depression and may comprise the following elements. This list of possible constituent elements is intended to be exemplary only, and it is not intended that this list be used to limit the composition of the present application to just these elements. Persons having ordinary skill in the art relevant to the present disclosure may understand there to be equivalent elements that may be substituted within the present disclosure without changing the essential function or operation of the composition.

The various elements of the present disclosure may be related in the following exemplary fashion. It is not intended to limit the scope or nature of the relationships between the various elements, and the following examples are presented as illustrative examples only.

By way of example, some embodiments of the present disclosure include a method and composition for the treatment of treatment resistant depression, wherein the method comprises administering a therapeutically effective dosage of a single administration composition to a person in need, wherein the composition comprises a serotonin and norepinephrine reuptake inhibitor (SNRI), a tetracyclic antidepressant (TeCA), and a mood stabilizer, and wherein a therapeutically effective dosage may be a dosage that, when taken as directed, provides the desired results. The combined dosages may vary in amount and administration.

In a particular embodiment, the SNRI may comprise venlafaxine, the TeCA may comprise mirtazapine, and the mood stabilizer may comprise lithium.

In an alternate embodiment, the composition may comprise the SNRI and the TeCA without the mood stabilizer. As such, some embodiments may comprise venlafaxine and mirtazapine without the lithium components.

In embodiments, the single administration composition may be made using a tablet or extended-release capsule with venlafaxine, orally disintegrating (dissolving) tablet or oral immediate-release tablet for mirtazapine, and regular tablets or slow-release tablets for lithium mixed together and provided as a single tablet, capsule, oral disintegrating tablet, oral disintegrating strip, oral dispersible films, liquid solutions, extended release preparations, controlled release forms, lozenges, films, nasal sprays, patches, and the like. The single administration may result in only a single administration form, such as a single table, needing to be administered to a user to provide a therapeutically effective dosage.

In embodiments, the composition may comprise, for example, about 0.1 to about 75 weight % (wt. %) SNRI, about 0.6 to about 80 wt. % TeCA, and about 1 to about 99 wt. % mood stabilizer. For example, in a particular embodiment, the composition may comprise from about 1 to about 450 mg venlafaxine, about 1 to about 60 mg mirtazapine, and about 1 to about 1800 mg lithium. The composition may be administered in a daily formulation to prevent or reduce suicidal ideation, as measured by the Hamilton Depression Rating Scale, and prevent disease progression development of tolerance toward antidepressants.

The combination of venlafaxine, mirtazapine, and lithium in a single pill composition may prevent disease progression/modifying the course of depression, delay/prevent relapse or recurrence of depression, prevent the development of delusional/psychotic depression, be protective/remedy the development of tolerance toward the antidepressant, and provide a neuroprotective effect. The treatment may also provide a more effective treatment than conventional treatments, may increase the response rate to treatment, and may treat the residual symptoms of depression. In fact, combining venlafaxine, mirtazapine, and lithium into a single pill may result in a synergistic combination, wherein the combination works better than administering the ingredients independently to the patient in need.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A composition for the treatment of treatment resistant depression, the composition comprising:
a serotonin and norepinephrine reuptake inhibitor (SNRI); and
a tetracyclic antidepressant (TeCA),
wherein the composition is in a single administrable form.

2. A composition for the treatment of treatment resistant depression, the composition comprising:
a serotonin and norepinephrine reuptake inhibitor (SNRI); and
a tetracyclic antidepressant (TeCA); and
a mood stabilizer.

3. The composition of claim 2, wherein:
the SNRI comprises venlafaxine;
the TeCA comprises mirtazapine; and
the mood stabilizer comprises lithium.

4. The composition of claim 2, wherein the composition comprises:
about 0.1 to about 75 weight % (wt. %) SNRI;
about 0.6 to about 80 wt. % TeCA, and
about 1 to about 99 wt. % mood stabilizer.

5. The composition of claim 1, wherein the composition is formed into a member selected from the group consisting of a single dosage pill and a single dosage tablet.

6. A method of treating treatment resistant depression, the method comprising:
administering a therapeutically effective dosage of a composition to a patient in need,
wherein the composition comprises:
a serotonin and norepinephrine reuptake inhibitor (SNRI); and
a tetracyclic antidepressant (TeCA), and
a mood stabilizer.

7. The method of claim 6, wherein:
the SNRI comprises venlafaxine;
the TeCA comprises mirtazapine; and
the mood stabilizer comprises lithium.

8. The method of claim 7, wherein the composition comprises:
about 1 to about 450 mg venlafaxine;
about 1 to about 60 mg mirtazapine; and
about 1 to about 1800 mg lithium.

9. The method of claim 6, wherein the composition is combined into a member selected from the group consisting of a single dosage pill and a single dosage tablet.
